(19) United States
(12) Patent Application Publication
(10) Pub. No.: US 2019/0009110 A1
(43) Pub. Date: Jan. 10, 2019

(54) ULTRASOUND ENERGY APPLICATOR

(71) Applicant: SLENDER MEDICAL LTD., Herziliya (IL)

(72) Inventors: Yossi GROSS, Moshav Mazor (IL); Ilan PASTERNAK, Netaim (IL)

(73) Assignee: SLENDER MEDICAL LTD., Herziliya (IL)

(21) Appl. No.: 15/869,376

(22) Filed: Jan. 12, 2018

Related U.S. Application Data
(60) Provisional application No. 62/529,112, filed on Jul. 6, 2017.

(51) Int. Cl. A61N 7/02

(52) U.S. Cl. CPC A61N 7/02; A61N 2007/0034

(57) ABSTRACT

Apparatus is provided for use with skin of a subject, the apparatus including a handheld unit and an ultrasound transducer disposed in the handheld unit and configured to transmit ultrasound energy into the skin. An outwardly-facing surface of the handheld unit includes a transducer-skin interface that is configured (a) to contact the skin when the handheld unit is positioned against the skin and (b) to slide against the skin. A sensor is disposed in the handheld unit and is configured to detect a distance between the transducer-skin interface to a bone under the skin of the subject when the transducer-skin interface is positioned against the skin. Control circuitry is configured to drive the ultrasound transducer to apply the ultrasound energy into the skin at different respective power levels according to the detected distance.

### CROSS REFERENCE TO RELATED APPLICATIONS

The present application claims the priority of U.S. 62/529,112 to Gross, filed Jul. 6, 2017, entitled, “Ultrasound energy applicator,” which is incorporated herein by reference.

### FIELD OF THE INVENTION

The present invention relates generally to skin treatment and particularly to methods and apparatus for skin rejuvenation by application of ultrasound energy.

### BACKGROUND

Skin, the body's largest organ, is composed of multiple layers. The outer layer, epidermis, is divided into several sublayers. Beneath the epidermis lies the dermis skin layer, which is composed of two layers, the upper papillary layer and the lower reticular layer.

A major structural component of the dermis skin layer is collagen, a fibrous protein, which contributes to skin strength and elasticity. As such, collagen formation, and in contrast, age-related collagen decline, leads to changes in mechanical properties of the skin, such as texture and resilience. Thermal treatment of the skin can lead to thermal shrinkage of collagen, which occurs by the dissociation of heat-sensitive bonds of the collagen molecule. Thermal denaturing of collagen typically results in a tightening effect of the skin.

Visible effects of aging or damage of the skin are disturbing to many individuals and therefore methods for rejuvenation of maturing or damaged skin are of interest. Some skin rejuvenation methods include application of energy to heat selected areas of the skin in order to change the appearance of the treated skin.

US 2013/0338545 to Azhari et al., which is incorporated herein by reference, describes apparatus including a skin-application portion, configured to move across skin of a subject. At least one acoustic element is coupled to the skin-application portion and configured to be placed in acoustic contact with the skin, and configured to apply ultrasound energy to the skin. The apparatus additionally includes circuitry configured to generate a current responsive to motion of the skin-application portion; and a control unit, which is configured to receive the current, to determine, responsive thereto, whether the skin-application portion is moving with respect to the skin, and to drive the acoustic element to apply the ultrasound energy to the skin responsive to determining that the skin-application portion is moving with respect to the skin.

US 2016/0375271 to Tsoref et al., which is incorporated herein by reference, describes a cartridge apparatus for use with a handle. The apparatus includes a housing which: (a) includes a handle-coupling portion shaped and sized to securely couple the housing to the handle and to facilitate separation of the housing from the handle, (b) is shaped to define a skin-application portion on an outer surface of the housing, and (c) includes at least one ultrasound element in acoustic communication with the skin-application portion and configured to transmit ultrasound energy to the skin through the skin-application portion when the cartridge is attached to the handle. The cartridge apparatus also includes an inflatable element inflated with a fluid, the fluid being in fluid communication with the skin-application portion. Other applications are also described.

US 2016/0375271 to Tsoref further describes the apparatus as being typically configured to verify sufficient acoustic contact of skin-application portion with the skin, by receiving an echo of transmitted ultrasound waves. For such applications, the ultrasound element also functions as a receiver, receiving the echo of the transmitted energy to determine whether the ultrasound energy is aimed at a bony structure underlying a skin surface (e.g., a cheek bone) or soft tissue (e.g., the eye). As described, typically, when the apparatus determines that the ultrasound energy is being transmitted toward soft tissue, e.g., the eye, the control unit inhibits the ultrasound element from transmitting treatment energy, e.g., high intensity focused ultrasound (HIFU) energy, to the skin.

US 2011/0251524 to Azhari, which is incorporated herein by reference, describes apparatus including first and second support structures for placement on skin of a subject. At least one of the first and second structures is moveable with respect to the other so as to draw a portion of the skin and underlying tissue of the subject between respective lateral surfaces of the support structures. At least one ultrasound transducer is moveably coupled to the first support structure along an axis of the structure that is not parallel to the lateral surface of the structure. The ultrasound transducer is configured to transmit toward the portion of skin and underlying tissue one or more forms of acoustic radiation energy, including treatment energy. At least one acoustic element is coupled to the second support structure. Other applications are also described.

### SUMMARY OF THE INVENTION

A handheld unit for use with skin of a subject is provided. The handheld unit typically has a proximal and distal end with an ultrasound transducer, configured to transmit ultrasound energy into the skin, disposed in the distal end. A transducer-skin interface, disposed on an outwardly-facing surface of the handheld unit, e.g., an outwardly facing surface of the distal end, is configured to contact the skin when the handheld unit is placed against the skin and to slide against the skin. The transducer-skin interface comprises a rigid ultrasound-conducting plastic that is substantially impermeable to water and substantially transparent (i.e., providing at least 85% transmission) to ultrasound energy at a transmission level of 10 J/s/cm2. Control circuitry in the handheld unit drives the ultrasound transducer to apply the ultrasound energy to the skin.

In accordance with some applications of the present invention, the ultrasound transducer comprises a piezoelectric crystal assembly that is shaped to focus the ultrasound energy in a focal line. Control circuitry in the handheld unit drives the ultrasound transducer to apply the ultrasound energy to the skin such that tissue 2-3.5 mm under the skin is heated in discrete lines as the transducer-skin interface slides against the skin.

In accordance with some applications of the present invention, a sensor is disposed in the handheld unit and configured to detect a distance between the transducer-skin interface to a bone under the skin of the subject when the transducer-skin interface is positioned against the skin. The control circuitry is configured to drive the ultrasound transducer to apply the ultrasound energy into the skin at different respective power levels according to the detected distance.

In accordance with some applications of the present invention, cooling is provided to the skin by a cooling element that is inlaid into a transducer-side of the transducer-skin interface such that a skin-side of the transducer-skin interface is uninterrupted by the cooling element.

In accordance with some applications of the present invention, the handheld unit is shaped to define a chamber, such that the rigid transducer-skin interface comprises a side of the chamber and the ultrasound transducer is disposed within the chamber. A fluid is disposed within the chamber and a heat sink is disposed either in the handheld unit, or on a surface of the handheld unit. The fluid is cooled by a thermoelectric cooling element that is disposed in the handheld unit between the chamber and the heat sink, such that a cold side of the thermoelectric cooling element is in thermal contact with the chamber, and a hot side of the thermoelectric cooling element is in thermal contact with the heat sink. The cooled fluid in turn cools the skin through the rigid transducer-skin interface, as well as the ultrasound transducer itself.

In accordance with some applications of the present invention, a viscous substance is provided for use with the skin, and a sensor is disposed in the handheld unit. The sensor detects the presence of the viscous substance on the skin when the handheld unit is positioned against the skin. The control circuitry is configured to drive the ultrasound transducer to vary the power level of the ultrasound energy according to the detected presence of the viscous substance. For some applications, after the viscous substance is applied to the skin, an ultrasound conducting gel may be applied to the skin on top of the viscous substance. A user then places the handheld unit against the skin and the control circuitry is activated to vary the power level of the ultrasound energy according to the detected presence of the viscous substance as the user slides the handheld unit against the skin.

In accordance with some applications of the present invention, an ultrasound conducting treatment cream is provided for use with the skin. The ultrasound transducer may enhance diffusion of the treatment cream into the skin by transmitting the ultrasound energy through the treatment cream into the skin. For some applications, the control circuitry is configured to drive the ultrasound transducer to function in two modes, where the first mode is a low-power mode for diffusion-enhancement of the treatment cream into the skin and the second mode is a high-power therapeutic mode for transmitting therapeutic ultrasound into the skin.

There is therefore provided, in accordance with some applications of the present invention, apparatus for use with skin of a subject, the apparatus including:

a handheld unit;

an ultrasound transducer disposed in the handheld unit, and configured to transmit ultrasound energy into the skin,
an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin,

a sensor disposed in the handheld unit and configured to detect a distance between the transducer-skin interface to a bone under the skin of the subject when the transducer-skin interface is positioned against the skin; and

control circuitry configured to drive the ultrasound transducer to apply the ultrasound energy into the skin at different respective power levels according to the detected distance.

For some applications, the transducer-skin interface includes a rigid transducer-skin interface.

For some applications, the rigid transducer-interface includes a rigid ultrasound-conducting plastic that is substantially (a) impermeable to water, and (b) transparent to ultrasound energy at a transmission level of 10 J/s/cm2.

For some applications, the ultrasound transducer is configured to transmit ultrasound energy at a range of power levels, the range of power levels including at least one power level between 10 W and 20 W and at least one power level between 25 W and 35 W.

For some applications, the control circuitry is configured to drive the ultrasound transducer to transmit the ultrasound energy at a low-power level of 10-20 W in response to the detected distance between the transducer-skin interface and a bone under the skin being less than a threshold distance, when the handheld unit is positioned against the skin.

For some applications, the threshold distance is 1-2 mm.

For some applications, the threshold distance is 2-4 mm.

For some applications, the control circuitry is configured to drive the ultrasound transducer to transmit the ultrasound energy at a high-power level of 20-35 W in response to the detected distance between the transducer-skin interface and a bone under the skin being greater than the threshold distance when the handheld unit is positioned against the skin.

For some applications, the handheld unit is configured to operate in at least three distinct modes of operation, the handheld unit further including a user control configured to activate the three distinct modes of operation:

(a) in a first mode of operation the control circuitry is configured to drive the ultrasound transducer to transmit the ultrasound energy at the low-power level of 10-20 W,

(b) in a second mode of operation the control circuitry is configured to drive the ultrasound transducer to transmit the ultrasound energy at the high-power level of 20-35 W, and

(c) in a third mode of operation the control circuitry is configured to automatically drive the ultrasound transducer to transmit the ultrasound energy at the different respective power levels according to the detected distance between the transducer-skin interface and a bone under the skin when the handheld unit is positioned against the skin.

For some applications, (a) the apparatus further includes a movement sensor configured to generate a speed signal indicative of the speed of the handheld unit when the handheld unit is in motion, and (b) the control circuitry is configured to receive the speed signal and to drive the ultrasound transducer to apply the ultrasound energy into the skin at different respective power levels according to the speed of the handheld unit.

For some applications, (a) the apparatus further includes an actuator configured to move the ultrasound transducer with respect to the transducer-skin interface, along a path that is normal to the skin when the handheld unit is positioned against the skin, and (b) the control circuitry is configured to activate the actuator to move the ultrasound transducer with respect to the transducer-skin interface in response to the detected distance between the transducer-skin interface and a bone under the skin.

For some applications, the control circuitry is configured to activate the actuator to move the ultrasound transducer away from the transducer-skin interface in response to the detected distance between the transducer-skin interface and a bone under the skin being less than a threshold distance, when the handheld unit is positioned against the skin.

For some applications, the threshold distance is 1-2 mm.

For some applications, the threshold distance is 2-4 mm.

For some applications. the control circuitry is configured to activate the actuator to move the ultrasound transducer toward the transducer-skin interface in response to the detected distance between the transducer-skin interface and a bone under the skin being greater than the threshold distance.

For some applications, the sensor includes a mechanical sensor configured to detect the distance between the transducer-skin interface and a bone under the skin by measuring a compliance of the skin when the handheld unit is positioned against the skin.

For some applications:

(a) the sensor includes the ultrasound transducer and is configured (i) to transmit one or more pulses of pulse-echo ultrasound energy into the skin at a power level of 0.1-5 W, and (ii) to receive a reflection of the transmitted pulse-echo ultrasound energy,

(b) the ultrasound transducer is configured to transmit therapeutic ultrasound energy into the skin at a power level of 10-35 W, and

(c) the control circuitry is configured (i) to determine a parameter indicative of the distance between the transducer-skin interface and a bone under the skin according to a time of flight between the transmitted and received pulses of pulse-echo ultrasound energy from the ultrasound transducer, and (ii) to drive the ultrasound transducer to subsequently apply the therapeutic ultrasound energy into the skin at different respective power levels according to the determined parameter.

For some applications, the ultrasound transducer is configured to transmit the therapeutic ultrasound energy into the skin in separate respective applications of the therapeutic ultrasound energy, and the control circuitry is configured to drive the ultrasound transducer to transmit the one or more pulses of pulse-echo ultrasound energy into the skin prior to transmitting at least one of the respective application of the therapeutic ultrasound energy into the skin.

For some applications, the control circuitry is configured to drive the ultrasound transducer to transmit the one or more pulses of pulse-echo ultrasound energy into the skin prior to transmitting each respective application of the therapeutic ultrasound energy into the skin.

For some applications, the apparatus further includes a motion sensor, and the control circuitry is configured to drive the ultrasound transducer to transmit the one or more pulses of pulse-echo ultrasound energy into the skin upon the motion sensor detecting at least 0.5 mm of motion, when the handheld unit is positioned against the skin.

For some applications, (a) the apparatus further includes an actuator configured to move the ultrasound transducer with respect to the transducer-skin interface, along a path that is normal to the skin when the handheld unit is positioned against the skin, and (b) the control circuitry is configured to activate the actuator to move the ultrasound transducer with respect to the transducer-skin interface in response to the determined parameter.

For some applications:

(a) the ultrasound transducer is a first ultrasound transducer configured to transmit therapeutic ultrasound energy into the skin at a power level of 10-35 W,

(b) the sensor includes a second ultrasound transducer configured to transmit pulse-echo ultrasound energy into the skin, and to receive a reflection of the transmitted pulse-echo ultrasound energy, and

(c) the control circuitry is configured (i) to determine a parameter indicative of the distance between the transducer-skin interface and a bone under the skin according to a time of flight between the transmitted and received pulse-echo ultrasound energy from the second ultrasound transducer, and (ii) to drive the first ultrasound transducer to transmit the ultrasound energy into the skin at different respective power levels according to the determined parameter.

For some applications, (a) the apparatus further includes an actuator configured to move the first ultrasound transducer with respect to the transducer-skin interface, along a path that is normal to the skin when the handheld unit is positioned against the skin, and (b) the control circuitry is configured to activate the actuator to move the first ultrasound transducer with respect to the transducer-skin interface in response to the determined parameter.

For some applications, the first ultrasound transducer is configured to transmit the therapeutic ultrasound energy at a first frequency of 4-6 MHz.

For some applications, the second ultrasound transducer is configured to transmit the pulse-echo ultrasound energy at a second frequency of 1-15 MHz.

For some applications, the second ultrasound transducer is configured to transmit the pulse-echo ultrasound energy at a second frequency, a ratio of the first frequency to the second frequency being 0.25-7.

For some applications, the second ultrasound transducer is configured to transmit the pulse-echo ultrasound energy at a power level of 0.1-5 W.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin,
an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin; and

a cooling element inlaid into a transducer-side of the transducer-skin interface such that a skin-side of the transducer-skin interface is uninterrupted by the cooling element.

For some applications, the transducer-skin interface includes a rigid transducer-skin interface.

For some applications, the rigid transducer-skin interface includes a rigid ultrasound-conducting plastic that is substantially (a) impermeable to water and (b) transparent to ultrasound energy at a transmission level of 10 J/s/cm2.

For some applications, the skin-side of the rigid transducer-skin interface includes an uninterrupted layer of the ultrasound-conducting plastic.

For some applications, a thickness of the rigid transducer-skin interface between the cooling element and the skin-side of the rigid transducer-skin interface is 0.1-0.6 mm.

For some applications, a thickness of the rigid transducer-skin interface that is not between the cooling element and the skin-side of the rigid transducer-skin interface is 0.3-2 mm.

For some applications, a ratio of (a) a thickness of the rigid transducer-skin interface that is not between the cooling element and the skin-side of the rigid transducer-skin interface is to (b) a thickness of the rigid transducer-skin interface between the cooling element and the skin-side of the rigid transducer-skin interface is 1-4.

For some applications, the cooling element includes a thermoelectric cooling element.

For some applications, the apparatus further includes a thermoelectric cooling element disposed in the handheld unit such that a cold side of the thermoelectric cooling element is in thermal contact with the inlaid cooling element.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin,
(a) an outwardly-facing surface of the handheld unit including a rigid transducer-skin interface that is configured (i) to contact the skin when the handheld unit is positioned against the skin such that the ultrasound transducer transmits the ultrasound energy through the rigid transducer-skin interface into the skin and (ii) to slide against the skin, and
(b) the handheld unit shaped to define a chamber, such that the rigid transducer-skin interface includes a side of the chamber and the ultrasound transducer is disposed within the chamber;

a fluid disposed within the chamber;

a heat sink disposed (a) in the handheld unit, or (b) on a surface of the handheld unit;

a thermoelectric cooling element disposed in the handheld unit between the chamber and the heat sink, such that a cold side of the thermoelectric cooling element is in thermal contact with the chamber, and a hot side of the thermoelectric cooling element is in thermal contact with the heat sink,
the thermoelectric cooling element configured to cool the fluid, which in turn cools (a) the skin through the rigid transducer-skin interface, and (b) the ultrasound transducer.

For some applications, the fluid includes distilled water and at least one additive material selected from the group consisting of: 40-60% ethylene glycol, 40-60% glycerol, and an antifoaming agent.

For some applications, the fluid includes an ultrasound conducting gel.

For some applications, the rigid transducer-skin interface includes a rigid ultrasound-conducting plastic that is substantially (a) impermeable to water and (b) transparent to ultrasound energy at a transmission level of 10 J/s/cm2.

For some applications, a thickness of the rigid transducer-skin interface at an interface between the rigid transducer-skin interface and the fluid is 0.1-0.6 mm.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin,
an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin;

a viscous substance for use with the skin;

a sensor disposed in the handheld unit, configured to detect presence of the viscous substance on the skin when the handheld unit is positioned against the skin; and

control circuitry configured to drive the ultrasound transducer to vary a power level of the ultrasound energy according to the detected presence of the viscous substance.

For some applications, the transducer-skin interface includes a rigid transducer-skin interface.

For some applications, the rigid transducer-interface includes a rigid ultrasound-conducting plastic that is substantially (a) impermeable to water, and (b) transparent to ultrasound energy at a transmission level of 10 J/s/cm2.

For some applications, the viscous substance includes magnetic particles and the sensor includes a magnetic sensor configured to detect the presence of the magnetic particles.

For some applications, the viscous substance includes a dye and the sensor includes an optical sensor configured to detect the dye.

For some applications, the dye includes a fluorescent dye.

For some applications, the apparatus further includes an ultrasound conducting gel for use with the skin, (a) a viscosity of the viscous substance is higher than a viscosity of the ultrasound conducting gel, and (b) the control circuitry is configured to drive the ultrasound transducer to transmit the ultrasound energy through the ultrasound conducting gel and to terminate transmission of the ultrasound energy in response to the presence of the viscous substance being detected on the skin.

For some applications, the viscosity of the viscous substance is at least 1.5 times higher than the viscosity of the ultrasound conducting gel.

For some applications, the apparatus further includes an ultrasound conducting gel for use with the skin, (a) a viscosity of the viscous substance is higher than a viscosity of the ultrasound conducting gel, and (b) the control circuitry has a mode in which the control circuitry is configured to drive the ultrasound transducer to transmit the ultrasound energy through the ultrasound conducting gel only in response to the presence of the viscous substance being detected on the skin.

For some applications, the viscosity of the viscous substance is at least 1.5 times higher than the viscosity of the ultrasound conducting gel.

For some applications, the ultrasound transducer is configured to transmit ultrasound energy at a range of power levels, the range of power levels including at least one power level between 10 W and 20 W and at least one power level between 25 W and 35 W.

For some applications, the control circuitry is configured to drive the ultrasound transducer to transmit the ultrasound energy at a high-power level of 20-35 W in response to the presence of the viscous substance being detected on the skin when the handheld unit is positioned against the skin.

For some applications, the control circuitry is configured to drive the ultrasound transducer to transmit the ultrasound energy at a low-power level of 10-20 W in response to the presence of the viscous substance being detected on the skin when the handheld unit is positioned against the skin.

For some applications, the control circuitry is configured to drive the ultrasound transducer to terminate transmission of the ultrasound energy in response to the presence of the viscous substance being detected on the skin.

For some applications, the viscous substance comprises a skin treatment product.

For some applications, the viscous substance includes at least one vitamin.

For some applications, the viscous substance includes botulinum toxin.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin at different respective power levels,
an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin;

an ultrasound conducting treatment cream for use with the skin, the ultrasound transducer being configured to enhance diffusion of the treatment cream into the skin by transmitting the ultrasound energy through the treatment cream into the skin; and

control circuitry configured to drive the ultrasound transducer to transmit the ultrasound energy into the skin in two phases:
(a) in a diffusion-enhancement phase, the ultrasound transducer is configured to enhance diffusion of the treatment cream into the skin by transmitting the ultrasound energy at a first power level through the treatment cream into the skin, and
(b) in a therapeutic phase, the ultrasound transducer is configured to transmit therapeutic ultrasound into the skin at a second power level higher than the first power level.

For some applications, the transducer-skin interface includes a rigid transducer-skin interface.

For some applications, the rigid transducer-interface includes a rigid ultrasound-conducting plastic that is substantially (a) impermeable to water, and (b) transparent to ultrasound energy at a transmission level of 10 J/s/cm2.

For some applications. the ultrasound transducer is configured to transmit ultrasound energy at a range of power levels, the range of power levels including at least one power level between 10 W and 20 W and at least one power level between 25 W and 35 W.

For some applications, the control circuitry is configured to set the first power level to be 10-20 W.

For some applications, the control circuitry is configured to set the second power level to be 20-35 W.

For some applications, the ultrasound transducer is configured to enhance diffusion of the treatment cream into an epidermis layer of the skin by transmitting the ultrasound energy through the treatment cream into the epidermis.

For some applications, a duration of the therapeutic phase is limited to a duration that is less than 10 minutes per any treatment area on the skin and a duration of the diffusion-enhancement phase is not limited to a duration that is less than 10 minutes per treatment area.

For some applications, the application further includes a heating element configured to further enhance the diffusion of the treatment cream by applying heat to the skin at a temperature of 30-40 degrees Celsius during the diffusion-enhancement phase.

For some applications, the apparatus further includes a cooling element configured to cool the skin during the therapeutic phase.

For some applications, the treatment cream includes at least one vitamin.

For some applications, the treatment cream includes botulinum toxin.

There is further provided, in accordance with some applications of the present invention, a method for treating skin of a subject, the method including:

applying a viscous substance on a first area of the skin;

applying an ultrasound conducting gel on a second area of the skin;

placing a handheld unit against the skin such that the ultrasound conducting gel is between the skin and the handheld unit,
(a) the handheld unit including (i) an ultrasound transducer disposed in the handheld unit configured to transmit ultrasound energy into the skin at different respective power levels, and (ii) a sensor disposed in the handheld unit, configured to detect presence of the viscous substance on the skin when the handheld unit is positioned against the skin, and
(b) an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin;

activating control circuitry to drive the ultrasound transducer to vary the power level of the ultrasound energy according to the detected presence of the viscous substance; and

sliding the handheld unit against the skin.

For some applications, applying the ultrasound conducting gel on the second area of the skin includes applying the ultrasound conducting gel to the second area of the skin such that the first area is at least partially within the second area.

For some applications, activating the control circuitry includes activating the control circuitry to drive the ultrasound transducer to transmit the ultrasound energy through the ultrasound conducting gel and to terminate transmission of the ultrasound energy in response to the presence of the viscous substance being detected on the skin.

For some applications, activating the control circuitry includes activating the control circuitry to drive the ultrasound transducer to operate in a mode in which the ultrasound transducer transmits the ultrasound energy through the ultrasound conducting gel only in response to the presence of the viscous substance being detected on the skin.

For some applications, applying the viscous substance includes applying a viscous substance that includes magnetic particles, and placing the handheld unit includes placing a handheld unit wherein the sensor is a magnetic sensor configured to detect the presence of the magnetic particles.

For some applications, applying the viscous substance includes applying a viscous substance that includes a dye, and placing the handheld unit includes placing a handheld unit wherein the sensor is an optical sensor configured to detect the presence of the dye.

For some applications, applying the viscous substance includes applying a viscous substance having a viscosity that is higher than a viscosity of the ultrasound conducting gel.

For some applications, applying the viscous substance includes applying a viscous substance having a viscosity that is at least 1.5 times higher than a viscosity of the ultrasound conducting gel.

There is further provided, in accordance with some applications of the present invention, a method for treating skin of a subject, the method including:

applying an ultrasound conducting treatment cream to an area on the skin;

placing a handheld unit against the skin such that the ultrasound conducting treatment cream is between the skin and the handheld unit,
(a) the handheld unit including an ultrasound transducer disposed in the handheld unit, and configured to transmit ultrasound energy into the skin at different respective power levels, and
(b) an outwardly-facing surface of the handheld unit including a transducer-skin interface that is configured (a) to contact the skin when the handheld unit is positioned against the skin such that the ultrasound transducer transmits the ultrasound energy through the transducer-skin interface into the skin and (b) to slide against the skin;

activating control circuitry to (a) drive the ultrasound transducer to enhance diffusion of the treatment cream into the skin by transmitting the ultrasound energy into the skin at a first power level, and subsequently, (b) drive the ultrasound transducer to transmit therapeutic ultrasound energy into the skin at a second power level higher than the first power level.

For some applications, applying the ultrasound conducting treatment cream includes applying an ultrasound conducting treatment cream that includes at least one vitamin.

For some applications, applying the ultrasound conducting treatment cream includes applying an ultrasound conducting treatment cream that includes botulinum toxin.

For some applications, placing a handheld unit against the skin includes placing a handheld unit that includes an ultrasound transducer configured to transmit ultrasound energy at a range of power levels, the range of power levels including at least one power level between 10 W and 20 W and at least one power level between 25 W and 35 W.

For some applications, activating the control circuitry to drive the ultrasound transducer to enhance diffusion of the treatment cream into the skin includes activating the control circuitry to drive the ultrasound transducer to transmit the ultrasound energy into the skin at a low-power level of 10-20 W.

For some applications, activating the control circuitry to drive the ultrasound transducer to transmit therapeutic ultrasound energy into the skin includes activating the control circuitry to drive the ultrasound transducer to transmit the ultrasound energy into the skin at a high-power level of 20-35 W.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin,
an outwardly-facing surface of the handheld unit including a rigid transducer-skin interface that is configured to (a) contact the skin when the handheld unit is positioned against the skin such that the ultrasound transducer transmits the ultrasound energy through the rigid transducer-skin interface, and (b) slide against the skin,
   wherein the transducer-skin interface comprises a rigid ultrasound-conducting plastic that is substantially (a) impermeable to water and (b) transparent to ultrasound energy at a transmission level of 10 J/s/cm2; and
control circuitry configured to drive the ultrasound transducer to apply the ultrasound energy to the skin.

For some applications, the rigid ultrasound conducting plastic includes a copolyester resin.

For some applications, the rigid ultrasound conducting plastic includes a polyamide copolymer.

For some applications, the ultrasound transducer is configured to transmit ultrasound energy into the skin at a frequency of 4-6 MHz.

For some applications, the ultrasound transducer is configured to transmit ultrasound energy into the skin at 10-35 W.

For some applications, the apparatus includes a gel in contact with the ultrasound transducer and the rigid ultrasound-conducting plastic.

For some applications, the handheld unit is shaped to define a chamber, such that the rigid transducer-skin interface includes a side of the chamber and the ultrasound transducer is disposed within the chamber, the apparatus further including:

a fluid disposed within the chamber;

a heat sink disposed (a) in the handheld unit, or (b) on a surface of the handheld unit; and

a thermoelectric cooling element disposed in the handheld unit between the chamber and the heat sink, such that a cold side of the thermoelectric cooling element is in thermal contact with the chamber, and a hot side of the thermoelectric cooling element is in thermal contact with the heat sink,
the thermoelectric cooling element configured to cool the fluid, which in turn cools (a) the skin through the rigid transducer-skin interface, and (b) the ultrasound transducer.

For some applications, the fluid includes distilled water and at least one additive material selected from the group consisting of: 40-60% ethylene glycol, 40-60% glycerol, and an antifoaming agent.

For some applications, the fluid includes an ultrasound conducting gel.

For some applications, a thickness of the rigid transducer-skin interface at an interface between the rigid transducer-skin interface and the fluid is 0.1-0.6 mm.

For some applications, the ultrasound transducer includes a piezoelectric crystal assembly shaped to focus the ultrasound energy in a focal line, and the control circuitry is configured to drive the ultrasound transducer to apply the ultrasound energy to the skin such that the skin is heated in discrete lines.

For some applications, the ultrasound transducer is configured to focus the ultrasound energy at a depth of 2-3.5 mm below a surface of the skin when the transducer-skin interface is in contact with the skin.

For some applications, the handheld unit includes at least one proximity sensor configured to generate a signal in response to the transducer-skin interface being in contact with the skin, and the control circuitry is configured to receive the signal and in response thereto stop the ultrasound transducer from applying the ultrasound energy to the skin when the transducer-skin interface is not in contact with the skin.

For some applications, the handheld unit includes at least one motion sensor configured to generate a signal in response to the handheld unit being in motion, and the control circuitry is configured to receive the signal and in response thereto stop the ultrasound transducer from applying the ultrasound energy to the skin when the handheld unit is not in motion.

For some applications, the control circuitry includes a printed circuit board (PCB) and wherein the at least one motion sensor is coupled to the PCB.

For some applications, the at least one motion sensor is configured to generate a signal indicative of rotation of the handheld unit with respect to the skin.

For some applications, the control circuitry is configured to receive the signal indicative of the rotation and in response thereto stop the ultrasound transducer applying the ultrasound energy to the skin.

For some applications, the control circuitry (a) includes a rotation indicator and (b) is configured to receive the signal indicative of the rotation and in response thereto activate the rotation indicator to indicate to a user of the apparatus that the transducer-skin interface is rotating with respect to the skin.

For some applications, the rotation indicator includes an audible rotation indicator.

For some applications, the rotation indicator includes a vibrating rotation indicator.

For some applications, the at least one motion sensor includes a 3-dimensional position sensor.

For some applications, the at least one motion sensor includes a 6-dimensional position and orientation sensor.

For some applications:

the at least one motion sensor includes two accelerometers (a) disposed at two opposite sides of the handheld unit and (b) configured to generate two respective signals in response to each of the two opposite sides of the handheld unit accelerating with respect to the skin, and

the control circuitry is configured to receive the two respective signals and in response thereto (a) determine which of the two sides of the handheld unit is accelerating with respect to the skin more than the other one of the two sides is accelerating with respect to the skin and (b) regulate application of the ultrasound energy by the ultrasound transducer in response to the determining.

For some applications, the control circuitry is configured to pulse the ultrasound energy such that as the transducer-skin interface slides against the skin, the ultrasound energy heats tissue under the skin in discrete lines, wherein a space between the discrete lines is 1-2 mm.

For some applications, the handheld unit includes at least one movement sensor configured to generate a signal indicative of a speed of the transducer-skin interface sliding against the skin, and the control circuitry is configured to receive the signal indicative of the speed and, in response to the speed, regulate a length of time prior to heating a subsequent discrete line in the skin.

For some applications, the control circuitry includes a printed circuit board (PCB), and the at least one movement sensor is coupled to the PCB.

For some applications, the control circuitry includes an energy indicator and is configured to (a) drive the ultrasound transducer to apply the ultrasound energy to the skin at a transmission level of 5-15 J/s, (b) activate the energy indicator when an amount of energy applied to the skin reaches a threshold value, (c) detect motion of the transducer-skin interface with respect to the skin following the activating of the energy indicator, and (d) subsequently, drive the ultrasound transducer to apply additional ultrasound energy to the skin.

For some applications, the energy indicator includes an audible energy indicator.

For some applications, the energy indicator includes a vibrating energy indicator.

For some applications:

the ultrasound transducer is configured to apply the ultrasound energy to the skin at a plurality of different transmission levels, and

the handheld unit includes a user input, wherein activation of the user input determines at which of the plurality of transmission levels the ultrasound transducer applies the ultrasound energy to the skin.

For some applications, the apparatus further includes an actuator configured to move the ultrasound transducer with respect to the transducer-skin interface, and the control circuitry is configured to (a) drive the ultrasound transducer to apply a first application of ultrasound energy to the skin, (b) activate the actuator to move the ultrasound transducer with respect to the transducer-skin interface, and (c) subsequently drive the ultrasound transducer to apply a second application of ultrasound energy to the skin such that the skin is heated in discrete lines with 1-2 mm of space between the discrete lines.

For some applications, the piezoelectric assembly is shaped as a segment of a hollow cylinder and has a length of 6-10 mm along a longitudinal axis of the cylinder.

For some applications, the piezoelectric crystal assembly is shaped such that a width of the focal line is 60-100 microns.

For some applications, the piezoelectric crystal assembly includes exactly one piezoelectric crystal having a length of 6-10 mm such that the ultrasound energy has a continuous focal line having a length of 6-10 mm.

For some applications, the piezoelectric crystal assembly includes a plurality of piezoelectric crystals disposed along a length of 6-10 mm, such that the ultrasound energy has a dashed focal line having a length of 6-10 mm and comprising discrete portions.

For some applications, the handheld unit has a proximal end and a distal end, the ultrasound transducer is disposed in the distal end of the handheld unit, and the rigid transducer-skin interface is configured to contact the skin when the distal end of the handheld unit is positioned against the skin.

There is further provided, in accordance with some applications of the present invention, a method for treating skin of a subject, the method including:

placing a handheld unit against the skin of the subject, the handheld unit including an ultrasound transducer configured to emit ultrasound energy having a focal line; and

sliding the handheld unit against the skin, such that:
while the ultrasound transducer is at a first location with respect to the skin, the ultrasound transducer heats a first discrete line of the skin by applying ultrasound energy into the skin, and
subsequently, while the ultrasound transducer is at a second location with respect to the skin 1-2 mm from the first location, the ultrasound transducer heats a second discrete line of the skin by applying ultrasound energy into the skin.

For some applications, the apparatus further includes moving the ultrasound transducer from the first location to the second location.

There is further provided, in accordance with some applications of the present invention, apparatus for use with skin of a subject, the apparatus including:

a handheld unit;

an ultrasound transducer disposed in the handheld unit, and configured to transmit ultrasound energy into the skin, the ultrasound transducer including a piezoelectric crystal assembly shaped to focus the ultrasound energy in a focal line;

a transducer-skin interface configured to (a) contact the skin when the handheld unit is positioned against the skin and (b) slide against the skin; and

control circuitry configured to drive the ultrasound transducer to apply the ultrasound energy to the skin such that the skin is heated in discrete lines.

For some applications, the ultrasound transducer is configured to focus the ultrasound energy at a depth of 2-3.5 mm below a surface of the skin when the transducer-skin interface is in contact with the skin.

For some applications, the handheld unit includes at least one proximity sensor configured to generate a signal in response to the transducer-skin interface being in contact with the skin, and the control circuitry is configured to receive the signal and in response thereto stop the ultrasound transducer from applying the ultrasound energy to the skin when the transducer-skin interface is not in contact with the skin.

For some applications, the handheld unit includes at least one motion sensor configured to generate a signal in response to the handheld unit being in motion, and the control circuitry is configured to receive the signal and in response thereto stop the ultrasound transducer from applying the ultrasound energy to the skin when the handheld unit is not in motion.

For some applications, the control circuitry includes a printed circuit board (PCB) and wherein the at least one motion sensor is coupled to the PCB.

For some applications, the at least one motion sensor is configured to generate a signal indicative of rotation of the handheld unit with respect to the skin.

For some applications, the control circuitry is configured to receive the signal indicative of the rotation and in response thereto stop the ultrasound transducer applying the ultrasound energy to the skin.

For some applications, the control circuitry (a) includes a rotation indicator and (b) is configured to receive the signal indicative of the rotation and in response thereto activate the rotation indicator to indicate to a user of the apparatus that the transducer-skin interface is rotating with respect to the skin.

For some applications, the rotation indicator includes an audible rotation indicator.

For some applications, the rotation indicator includes a vibrating rotation indicator.

For some applications, the at least one motion sensor includes a 3-dimensional position sensor.

For some applications, the at least one motion sensor includes a 6-dimensional position and orientation sensor.

For some applications:

the at least one motion sensor includes two accelerometers (a) disposed at two opposite sides of the handheld unit and (b) configured to generate two respective signals in response to each of the two opposite sides of the handheld unit accelerating with respect to the skin, and

the control circuitry is configured to receive the two respective signals and in response thereto (a) determine which of the two sides of the handheld unit is accelerating with respect to the skin more than the other one of the two sides is accelerating with respect to the skin and (b) regulate application of the ultrasound energy by the ultrasound transducer in response to the determining.

For some applications, the control circuitry is configured to pulse the ultrasound energy such that as the transducer-skin interface slides against the skin, the ultrasound energy heats tissue under the skin in discrete lines, wherein a space between the discrete lines is 1-2 mm.

For some applications, the handheld unit includes at least one movement sensor configured to generate a signal indicative of a speed of the transducer-skin interface sliding against the skin, and wherein the control circuitry is configured to receive the signal indicative of the speed and, in response to the speed, regulate a length of time prior to heating a subsequent discrete line in the skin.

For some applications, the control circuitry includes a printed circuit board (PCB), and wherein the at least one movement sensor is coupled to the PCB.

For some applications, the control circuitry includes an energy indicator and is configured to (a) drive the ultrasound transducer to apply the ultrasound energy to the skin at a transmission level of 5-15 J/s, (b) activate the energy indicator when an amount of energy applied to the skin reaches a threshold value, (c) detect motion of the transducer-skin interface with respect to the skin following the activating of the energy indicator, and (d) subsequently, drive the ultrasound transducer to apply additional ultrasound energy to the skin.

For some applications, the energy indicator includes an audible energy indicator.

For some applications, the energy indicator includes a vibrating energy indicator.

For some applications:

the ultrasound transducer is configured to apply the ultrasound energy to the skin at a plurality of different transmission levels, and

the handheld unit includes a user input, wherein activation of the user input determines at which of the plurality of transmission levels the ultrasound transducer applies the ultrasound energy to the skin.

For some applications, the apparatus further includes an actuator configured to move the ultrasound transducer with respect to the transducer-skin interface, and the control circuitry is configured to (a) drive the ultrasound transducer to apply a first application of ultrasound energy to the skin, (b) activate the actuator to move the ultrasound transducer with respect to the transducer-skin interface, and (c) subsequently drive the ultrasound transducer to apply a second application of ultrasound energy to the skin such that the skin is heated in discrete lines with 1-2 mm of space between the discrete lines.

For some applications, the piezoelectric crystal assembly is shaped as a segment of a hollow cylinder and has a length of 6-10 mm along a longitudinal axis of the cylinder.

For some applications, the piezoelectric crystal assembly is shaped such that a width of the focal line is 60-100 microns.

For some applications, the piezoelectric crystal assembly includes exactly one piezoelectric crystal having a length of 6-10 mm such that the ultrasound energy has a continuous focal line having a length of 6-10 mm.

For some applications, the piezoelectric crystal assembly includes a plurality of piezoelectric crystals disposed along a length of 6-10 mm, such that the ultrasound energy has a dashed focal line having a length of 6-10 mm and including discrete portions.

For some applications, the handheld unit has a proximal end and a distal end, the ultrasound transducer is disposed in the distal end of the handheld unit, and the rigid transducer-skin interface is configured to contact the skin when the distal end of the handheld unit is positioned against the skin.

The present invention will be more fully understood from the following detailed description of applications thereof, taken together with the drawings, in which:

### BRIEF DESCRIPTION OF THE DRAWINGS

FIG. 1 is a schematic illustration of a handheld unit for use with skin of a subject, in accordance with some applications of the present invention;

FIGS. 2A-B are schematic illustrations of a piezoelectric crystal assembly, in accordance with some applications of the present invention;

FIG. 3 is a schematic illustration of ultrasound energy applied to the skin of the subject, in accordance with some applications of the present invention;

FIG. 4 is a schematic illustration of a transducer-skin interface in contact with the skin of the subject and an actuator coupled to an ultrasound transducer, in accordance with some applications of the present invention;

FIG. 5 is a schematic illustration of the handheld unit, in accordance with some applications of the present invention;

FIG. 6 is a schematic illustration of the transducer-skin interface in contact with the skin of the subject, an actuator coupled to the ultrasound transducer, and bone under the skin of the subject, in accordance with some applications of the present invention;

FIG. 7 is a schematic illustration of a piezoelectric crystal assembly in accordance with some applications of the present invention;

FIGS. 8A-B are schematic illustrations of the handheld unit showing a chamber that houses the ultrasound transducer, in accordance with some applications of the present invention;

FIGS. 9A-B are schematic illustrations of a wrinkle in the skin of the subject, a viscous substance applied in or around the wrinkle, and an ultrasound conducting gel in accordance with some applications of the present invention; and

FIG. 10 is a schematic illustration of an ultrasound conducting treatment cream on the skin of the subject, in accordance with some applications of the present invention.

### DETAILED DESCRIPTION

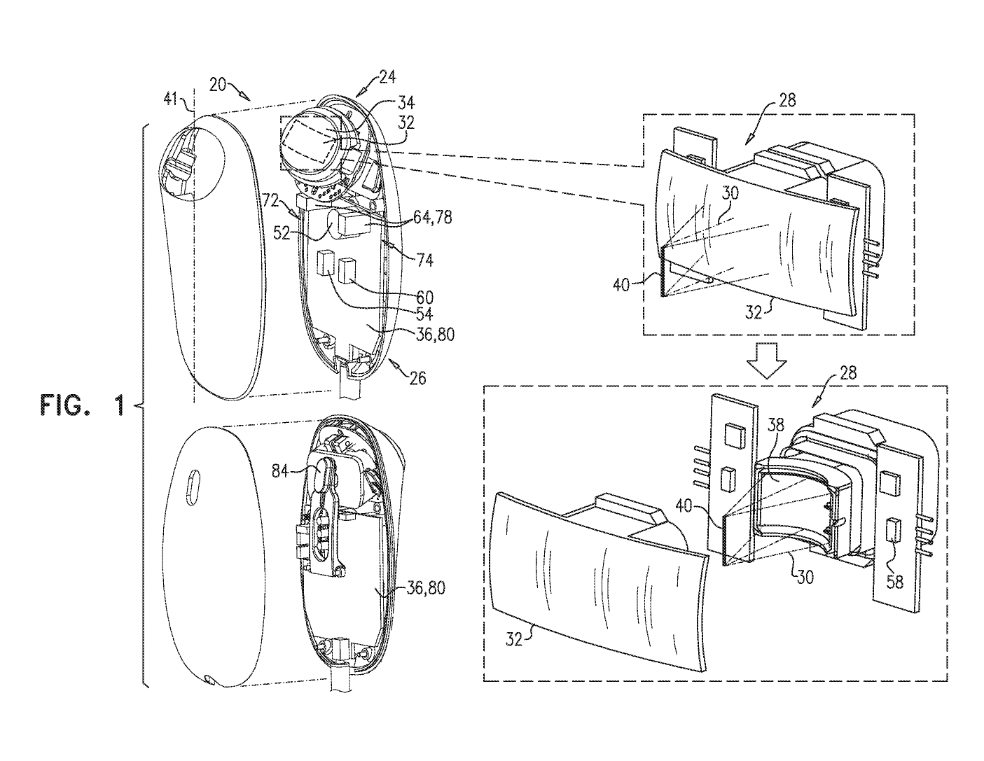

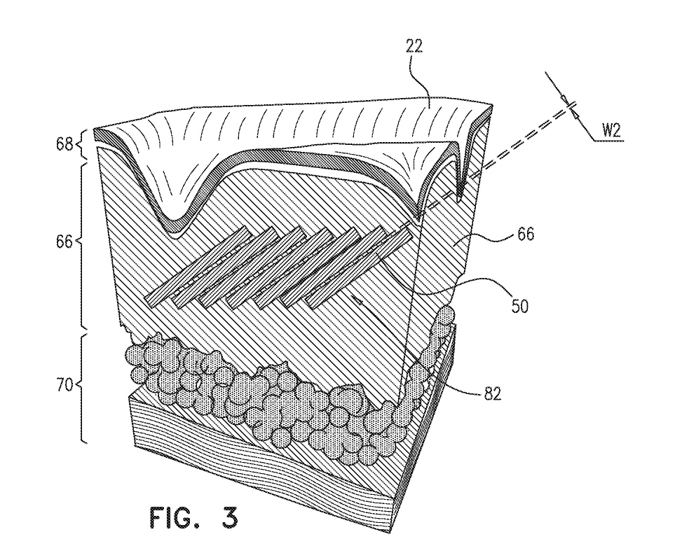

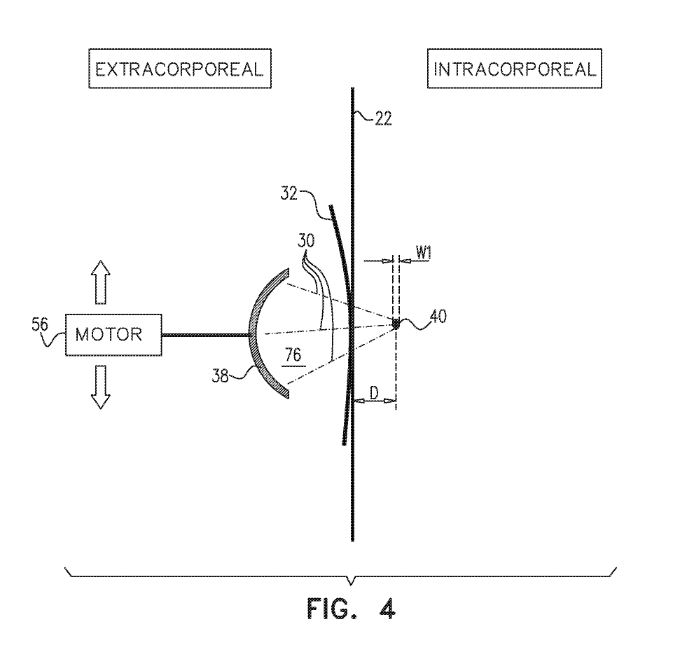

Reference is made to FIG. 1, which is a schematic illustration of a handheld unit 20 for use with skin 22 (FIG. 3) of a subject, in accordance with some applications of the present invention. Handheld unit 20 comprises a distal end 24 and a proximal end 26. An ultrasound transducer 28 is disposed in distal end 24 and is configured to transmit ultrasound energy 30 (FIG. 4), e.g., high intensity focused ultrasound (HIFU) energy into skin 22. An outwardly-facing surface 34 of handheld unit 20 comprises a transducer-skin interface 32. Transducer-skin interface 32 is configured to (a) contact skin 22 when handheld unit 20 is positioned against skin 22 and (b) slide against skin 22, such that ultrasound transducer 28 is in acoustic contact with skin 22. Control circuitry 36 is configured to drive ultrasound transducer 28 to apply ultrasound energy 30 to skin 22. Typically, ultrasound transducer 28 is configured to transmit ultrasound energy 30 into skin 22 at (a) a frequency of at least 4 MHz and/or less than 6 MHz and (b) at a range of power levels including at least one power level between 10 W and 20 W and at least one power level between 25 W and 35 W, e.g., at a power level at least 10 Watts and/or less than 35 Watts. Typically, ultrasound transducer 28 is configured to focus the ultrasound energy 30 at a depth D of at least 2.0 mm and/or less than 3.5 mm below a surface of skin 22 (FIG. 4).

The combination of high power ultrasound energy and a shallow focal depth may cause conventional rigid transducer-skin interfaces to heat up and deform. Use of a soft membrane as the transducer-skin interface may avoid this, however liquid surrounding the transducer, e.g., cooling liquid, can evaporate through the soft membrane. Applications of the present invention provide a rigid transducer-skin interface that comprises a rigid ultrasound-conducting plastic, such as a copolyester resin or polyamide copolymer, e.g., EMS-Grivory® Grilamid TR 55, or Eastman Tritan™ Copolyester TX1001, that is at least 0.3 mm and/or less than 0.5 mm thick, and that is substantially (a) impermeable to water, and (b) transparent to ultrasound energy at a transmission level of 10 J/s/cm2, i.e., the interface transmits at least 85% of the energy. Use of such a material for transducer-skin interface 32 provides handheld unit 20 with a longer shelf-life, e.g., longer than three years, due to decreased evaporation of water contained between ultrasound transducer 28 and transducer-skin interface 32, while simultaneously allowing high levels of ultrasound energy to pass through it without, for example, melting or otherwise deforming transducer-skin interface 32. For some applications, to further decrease evaporation, a gel 76, e.g., a hygroscopic gel, is used instead of water, gel 76 being in contact with both ultrasound transducer 28 and transducer-skin interface 32 (FIG. 4).

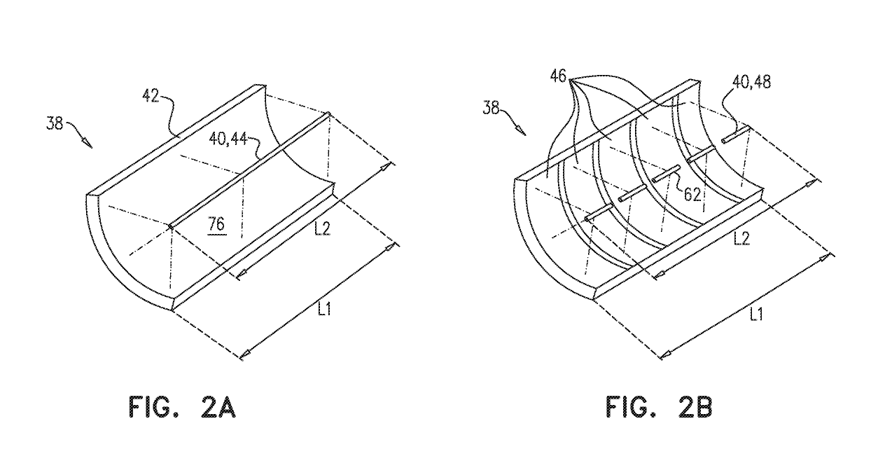

Reference is now made to FIGS. 2A-B, which are schematic illustrations of a piezoelectric crystal assembly, in accordance with some applications of the present invention. Ultrasound transducer 28 comprises a piezoelectric crystal assembly 38 that is shaped as a segment of a hollow cylinder, e.g., a circular cylinder, elliptical cylinder, or another cylindrical shape suitable for a HIFU transducer, in order to focus ultrasound energy 30 in a focal line 40, such as is shown in FIG. 2A. When handheld unit 20 is placed against skin 22 focal line 40 is typically substantially parallel to skin 22. Typically, (a) a length L1 of piezoelectric crystal assembly 38 is at least 6 mm and/or less than 10 mm, such that a length L2 of focal line 40 is at least 6 mm and/or less than 10 mm, and (b) piezoelectric crystal assembly 38 is shaped such that focal line 40 has a width W (FIG. 4) of at least 60 microns and/or less than 100 microns.

For some applications, piezoelectric crystal assembly 38 comprises exactly one piezoelectric crystal 42 having length L1, such that focal line 40 of ultrasound energy 30 is a continuous focal line 44 having length L2. For some applications, piezoelectric crystal assembly 38 comprises a plurality of piezoelectric crystals 46 disposed along length L1 (or is otherwise configured), such that focal line 40 of ultrasound energy 30 is a dashed focal line 48 having discrete portions 62 that make up length L2, such as is shown in FIG. 2B.

For some applications, such as is shown in FIG. 1, piezoelectric crystal assembly 38 is disposed in handheld unit 20 such that focal line 40 is substantially parallel to a long axis 41 of handheld unit 20. Alternatively, piezoelectric crystal assembly 38 may be disposed in handheld unit 20 such that focal line 40 is substantially perpendicular to long axis 41 of handheld unit 20.

Reference is now made to FIG. 3, which is a schematic illustration of ultrasound energy 30 applied to skin 22, in accordance with some applications of the present invention. Piezoelectric crystal assembly 38 being shaped to focus ultrasound energy 30 in a line allows skin 22 to be heated by ultrasound energy 30 in discrete lines 50. Typically, ultrasound transducer 28 is configured to focus the ultrasound energy 30 at a depth D of at least 2.0 mm and/or less than 3.5 mm below a surface of skin 22 (FIG. 4), such that typically only tissue 82 in a dermis layer 66 of under skin 22 is heated, and not epidermis 68 or subcutaneous 70 regions. Discrete lines of heating produce discrete areas of damaged tissue next to areas of undamaged tissue. It is herein hypothesized that undamaged cells help speed up the collagen regeneration process in the damaged tissue, thereby speeding up the treatment process.

Reference is again made to FIG. 1. For some applications, handheld unit 20 comprises at least one proximity sensor 58, e.g., an optical sensor, configured to generate a signal in response to transducer-skin interface 32 being in contact with skin 22. Control circuitry 36 receives the signal from sensor(s) 58 and in response thereto stops ultrasound transducer 28 from applying ultrasound energy 30 to skin 22 when transducer-skin interface 32 is not in contact with skin 22. Proximity sensor 58, for example, may be disposed on or near transducer-skin interface 32. Contact can also be sensed, for example, (a) by measuring pressure in gel 76 (e.g., when a membrane is used instead of a rigid transducer-skin interface 32), or (b) by measuring a reduction of reflection of ultrasound energy 30. Handheld unit 20 typically further comprises at least one motion sensor 78, e.g., an accelerometer, configured to generate a signal in response to handheld unit 20 being in motion. Control circuitry 36 receives the signal from motion sensor(s) 78 and in response thereto stops ultrasound transducer 28 from applying ultrasound energy 30 to skin 22 when handheld unit 20 is not in motion. For some applications, control circuitry 36 comprises a printed circuit board (PCB) 80, and motion sensor 78 is coupled to the PCB 80. Proximity sensor(s) 58 together with motion sensor(s) 78 prevent overheating of tissue 82 by control circuitry 36, thereby reducing application of ultrasound energy 30 to the skin while transducer-skin interface 32 is not moving along skin 22.

Handheld unit 20 is typically moved along skin 22 by the user. There is therefore a possibility of human error and it is possible that the user may accidentally rotate handheld unit 20 while ultrasound transducer 28 is applying ultrasound energy 30 to her skin. (It is noted that this rotation refers to rotation in a plane that is coplanar with skin 22.) If such a rotation occurs while ultrasound transducer 28 is applying ultrasound energy 30 to skin 22, it may result in ultrasound energy 30 being applied unevenly to skin 22. To avoid this, for some applications, motion sensor(s) 78 is configured to generate a signal indicative of rotation of handheld unit 20 with respect to skin 22. Control circuitry 36 receives the signal indicative of the rotation and in response thereto stops ultrasound transducer 28 from further applying ultrasound energy 30 to skin 22. For some applications, control circuitry 36 comprises a rotation indicator 60, e.g., an audible indicator or a vibrating indicator, and is configured to receive the signal indicative of the rotation and in response thereto to activate rotation indicator 60 in order to indicate to the user that she is rotating transducer-skin interface 32 with respect to her skin. For some applications, sensor(s) 58 comprises a 3-dimensional (3D) position sensor or a 6-dimensional position and orientation sensor.

For some applications, motion sensor(s) 78 comprise two accelerometers 64 respectively disposed at two opposite sides 72 and 74 of PCB 80 or elsewhere on or in handheld unit 20. Accelerometers 64 are configured to generate two respective signals in response to each of opposite sides 72 and 74 of PCB 80 accelerating with respect to skin 22. Control circuitry 36 receives the two respective signals and in response thereto (a) determines which of opposite sides 72 and 74 is accelerating with respect to skin 22 more than the other one of opposite sides 72 and 74 is accelerating with respect to skin 22, and (b) regulates the application of ultrasound energy 30 by ultrasound transducer 28. For example, control circuitry 36 may lower, e.g., stop, ultrasound transducer 28 applying ultrasound energy 30 from whichever side 72 or 74 is determined to be accelerating less.

For some applications, heating tissue 82 under skin 22 in discrete lines 50 is achieved by control circuitry 36 pulsing ultrasound energy 30 such that as transducer-skin interface 32 slides against skin 22, ultrasound energy 30 heats skin 22 in discrete lines 50 with a space W2 of at least 1 mm and/or less than 2 mm between discrete lines 50. For some applications, handheld unit 20 comprises at least one movement sensor 52 (FIG. 1) configured to generate a signal indicative of a speed of transducer-skin interface 32 sliding against skin 22. Control circuitry 36 receives the signal indicative of the speed and, in response thereto, regulates a length of time after heating a discrete line 50 in tissue 82 and prior to heating a subsequent discrete line 50 in tissue 82. Movement sensor(s) 52 may be coupled to PCB 80, or elsewhere on or in handheld unit 20.

Alternatively or additionally, discrete lines 50 of heating are achieved by control circuitry 36 having an energy indicator 54 (FIG. 1) and being configured to (a) drive ultrasound transducer 28 to apply ultrasound energy 30 to skin 22 at a transmission level of at least 5 J/s and/or less than 15 J/s, (b) activate energy indicator 54 when an amount of energy applied to skin 22 reaches a threshold value, (c) detect motion of transducer-skin interface 32 with respect to skin 22 following activation of energy indicator 54, and (d) subsequently, drive ultrasound transducer 28 to apply additional ultrasound energy 30 to skin 22. This way, a user places handheld unit 20 on her skin and waits for energy indicator 54 to be activated, indicating that at that location enough ultrasound energy 30 has been applied to her skin, she then slides handheld unit to a second location, at least 1 mm and/or less than 2 mm away from the first location, and control circuitry 36 will automatically drive ultrasound transducer 28 to apply additional ultrasound energy 30 to her skin until energy indicator 54 is activated again. Energy indicator 54 may be, for example, an audible indicator, or a vibrational indicator.

For some applications, ultrasound transducer 28 is configured to apply ultrasound energy 30 to tissue 82 at a plurality of different transmission levels. A user input 84 allows the user to choose a transmission level. A user may want to choose a higher or lower transmission level based on, for example, a specific treatment area, or user comfort.

Alternatively or additionally, a user may hold handheld unit 20 stationary against skin 22, and an actuator 56, e.g., a motor, (FIG. 4) moves ultrasound transducer 28 with respect to transducer-skin interface 32, e.g., along a path that is substantially parallel to skin 22, and substantially perpendicular to focal line 40. Control circuitry 36 is configured to (a) drive ultrasound transducer 28 to apply a first application of ultrasound energy 30 to skin 22, (b) activate actuator 56 to move ultrasound transducer 28 with respect to transducer-skin interface 32, and (c) subsequently drive ultrasound transducer 28 to apply a second application of ultrasound energy 30 to skin 22, such that skin 22 is heated in discrete lines 50 with at least 1 mm and/or less than 2 mm between them.

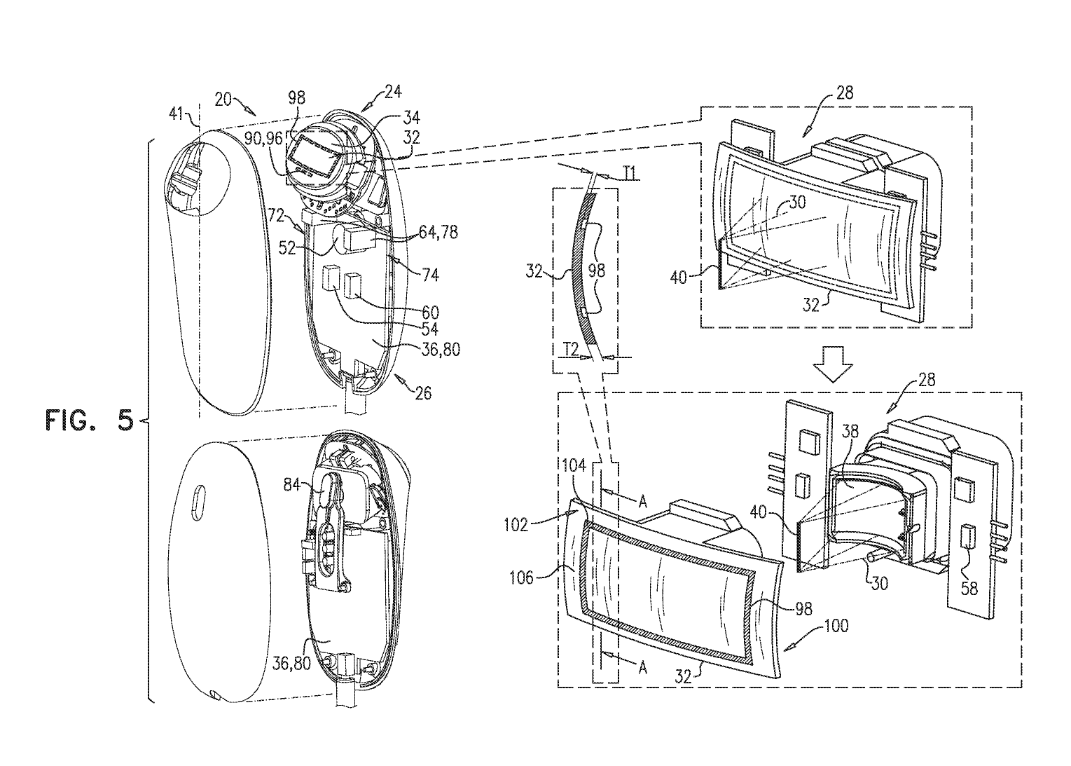

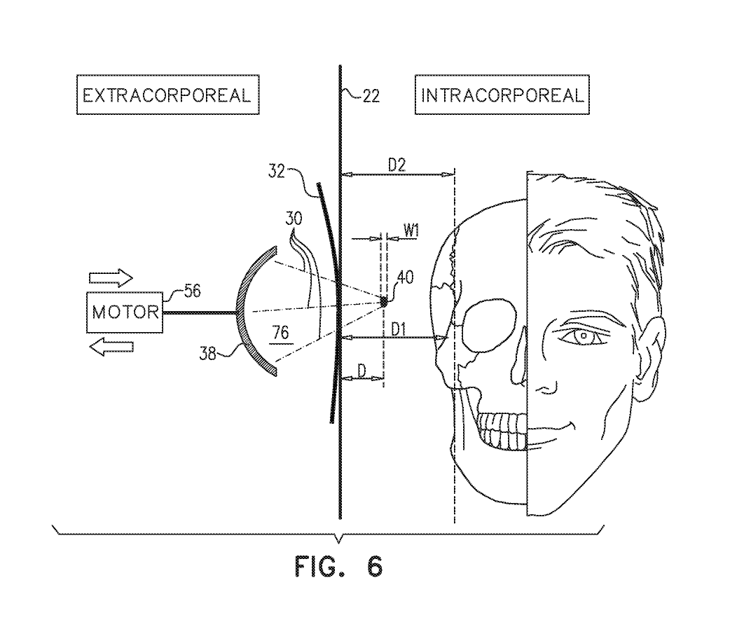

Reference is now made to FIG. 5, which is a schematic illustration of the handheld unit, in accordance with some applications of the present invention. As the user slides handheld unit 20 against skin 22, discomfort may occur if transducer-skin interface 32 applies energy to an area of skin 22 where there is a bone that is close to the surface of the skin. For some applications, a sensor 90 is disposed in handheld unit 20 and configured to detect a distance D1 (FIG. 6) between transducer-skin interface 32 and a bone 88 under skin 22 of the subj ect when transducer-skin interface 32 is positioned against skin 22. Control circuitry 36 is configured to drive ultrasound transducer 28 to apply ultrasound energy 30 into skin 22 at different respective power levels according to the detected distance D1 between transducer-skin interface 32 and bone 88.

As described hereinabove, ultrasound transducer 28 is typically configured to transmit ultrasound energy 30 into skin 22 at a range of power levels including at least one power level between 10 W and 20 W and at least one power level between 25 W and 35 W. For some applications, handheld unit 20 operates in at least three distinct modes. In a first mode of operation, control circuitry drives ultrasound transducer 28 to transmit ultrasound energy 30 into skin 22 at a low-power level of 10-20 W, and in a second mode of operation control circuitry 36 drives ultrasound transducer 28 to transmit ultrasound energy 30 into skin 22 at a high-power level of 20-30 W. A user input 84 may allow the user to manually choose between the first and second modes of operation. In a third mode of operation control circuitry 36 automatically drives ultrasound transducer 28 to transmit ultrasound energy 30 into skin 22 at the different respective power levels according to detected distance D1 between transducer-skin interface 32 and bone 88 under the skin.

For example, control circuitry 36 may be configured to (a) drive ultrasound transducer 28 to transmit ultrasound energy 30 into skin 22 at the low-power level of 10-20 W in response to detected distance D1 being less than a threshold distance D2 (FIG. 6), and (b) drive ultrasound transducer 28 to transmit ultrasound energy 30 into skin 22 at the high-power level of 20-35 W in response to detected distance D1 being greater than threshold distance D2. Threshold distance D2 may be 1-2 mm or 2-4 mm.

For some applications, control circuitry 36 may receive the signal indicative of the speed of handheld unit 20 from movement sensor 52 (as described hereinabove with reference to FIG. 1), and in response thereto drive ultrasound transducer 28 to apply ultrasound energy 30 into skin 22 at the different respective power levels according to the speed of handheld unit 20. For example, in order to maintain substantially even energy transmission per treatment area of skin 22, control circuitry 36 may be configured to (a) drive ultrasound transducer 28 to transmit ultrasound energy 30 into skin 22 at a higher power level in response to movement sensor 52 sensing an increase in speed of handheld unit 20, and (b) drive ultrasound transducer 28 to transmit ultrasound energy 30 into skin 22 at a lower power level in response to movement sensor 52 sensing a decrease in speed of handheld unit 20.

Reference is now made to FIG. 6, which is a schematic illustration of transducer-skin interface 32 in contact with skin 22 of the subject, an actuator coupled to ultrasound transducer 28, and bone 88 under skin 22 of the subject, in accordance with some applications of the present invention. For some applications, an actuator, e.g., a motor, such as actuator 56 as described hereinabove with reference to FIG. 4, is configured to move ultrasound transducer 28 with respect to transducer-skin interface 32 along a path that is normal to skin 22 when handheld unit 20 is positioned against the skin, thereby altering a depth D under the skin of the focus of the ultrasound energy. Alternatively or additionally to varying the power level of therapeutic ultrasound energy 30, control circuitry 36 is configured to activate actuator 56 to move ultrasound transducer 28 with respect to transducer-skin interface 32 in response to detected distance D1 between transducer-skin interface 32 and bone 88 under the skin. For example, control circuitry 36 may (a) activate actuator 56 to move ultrasound transducer 28 away from transducer-skin interface 32 in response to detected distance D1 being less than threshold distance D2, and (b) activate actuator 56 to move ultrasound transducer 28 toward transducer-skin interface 32 in response to detected distance D1 being greater than threshold distance D2.

Reference is again made to FIG. 5. For some applications, ultrasound transducer 28 acts as sensor 90. Ultrasound transducer 28 may be configured to (a) transmit one or more pulses of pulse-echo ultrasound energy into skin 22 at a power level of 0.1-5 W and receive a reflection of the transmitted pulse-echo ultrasound energy, and (b) transmit therapeutic ultrasound energy 30 into skin 22 at a power level of 10-35 W. Control circuitry 36 determines a parameter indicative of distance D1 between transducer-skin interface 32 and bone 88 under skin 22 according to a time of flight between the transmitted and received pulses of pulse-echo ultrasound energy, and subsequently drives ultrasound transducer 28 to apply therapeutic ultrasound energy 30 into skin 22 at different respective power levels according to the determined parameter.

For some applications, alternatively or additionally to varying the power level of therapeutic ultrasound energy 30, control circuitry 36 may be configured to activate actuator 56 to move ultrasound transducer 28 with respect to transducer-skin interface 32, in response to the determined parameter. For example, control circuitry 36 may (a) activate actuator 56 to move ultrasound transducer 28 away from transducer-skin interface 32 in response to the determined parameter being indicative of distance D1 being less than threshold distance D2, and (b) activate actuator 56 to move ultrasound transducer 28 toward transducer-skin interface 32 in response to the determined parameter being indicative of distance D1 being greater than threshold distance D2.

For some applications, ultrasound transducer 28 is configured to transmit therapeutic ultrasound energy 30 in separate respective applications. Control circuitry 36 may drive ultrasound transducer 28 to transmit the one or more pulses of pulse-echo ultrasound energy into skin 22 prior to transmitting at least one, e.g., prior to transmitting each, of the respective applications of therapeutic ultrasound energy 30 into skin 22.

For some applications, a motion sensor, such as motion sensor 78 in FIG. 1, detects motion of handheld unit 20. Control circuitry 36 may be configured to drive ultrasound transducer 28 to transmit the one or more pulses of pulse-echo ultrasound energy into skin 22 upon motion sensor 78 detecting at least 0.5 mm of motion, when handheld unit 20 is positioned against skin 22.

For some applications, instead of ultrasound transducer 28 transmitting both the pulse-echo ultrasound energy and therapeutic ultrasound energy 30, sensor 90 is a second ultrasound transducer 92 that is configured to transmit the pulse-echo ultrasound energy into skin 22 and to receive a reflection of the transmitted pulse-echo ultrasound energy. For some applications, second ultrasound transducer 92 is configured to transmit the pulse-echo ultrasound energy at a power level of 0.1-5 W. Control circuitry 36 determines a parameter indicative of distance D1 between transducer-skin interface 32 and bone 88 under skin 22 according to a time of flight between the transmitted and received pulses of pulse-echo ultrasound energy, and subsequently drives ultrasound transducer 28 to apply therapeutic ultrasound energy 30 into skin 22 at different respective power levels according to the determined parameter.

Typically, a ratio between (a) a first frequency at which first ultrasound transducer 28 is configured to transmit therapeutic ultrasound energy 30, and (b) a second frequency at which second ultrasound transducer 92 is configured to transmit the pulse-echo ultrasound energy is 0.25-7. For example, first ultrasound transducer 28 may transmit therapeutic ultrasound energy 30 at a frequency of 4-6 MHz, and second ultrasound transducer 92 may transmit the pulse-echo ultrasound energy at a frequency of 1-15 MHz.

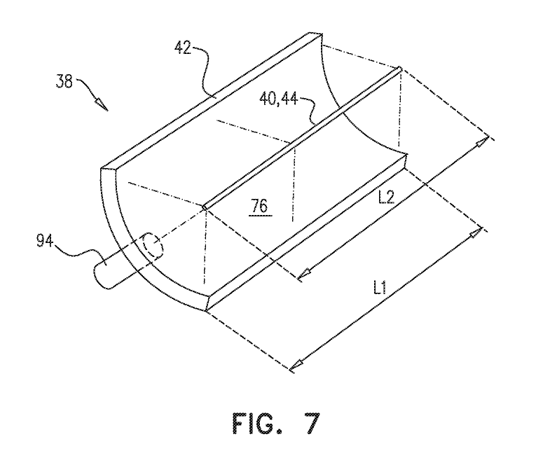

Reference is now made to FIG. 7, which is a schematic illustration of a piezoelectric crystal assembly in accordance with some applications of the present invention. For some applications, a piezoelectric crystal 94 of second ultrasound transducer 92 may be disposed adjacent to piezoelectric crystal assembly 38 of first ultrasound transducer 28. For some applications, such as when piezoelectric crystal assembly 38 of first ultrasound transducer 28 comprises a plurality of piezoelectric crystals 46 disposed along length L1 (such as is described hereinabove with reference to FIG. 2B), piezoelectric crystal 94 of second ultrasound transducer 92 may be disposed in a gap between two piezoelectric crystals 46 (configuration not shown).

Reference is again made to FIG. 5. For some applications sensor 90 is a mechanical sensor 96 configured to detect distance D1 between transducer-skin interface 32 and bone 88 under skin 22 by measuring a compliance of skin 22 when the handheld unit is positioned against the skin. For example, mechanical sensor 96 may comprise a plurality of prongs with each prong having a stress detector at a distal end thereof. At least one prong is shorter than the other prongs such that (a) when mechanical sensor 96 is pressed against an area of skin 22 where detected distance D1 is greater than threshold distance D2, all the prongs will detect a similar stress as mechanical sensor 96 is pressed into the soft tissue, whereas (b) when mechanical sensor 96 is pressed against an area of skin 22 where detected distance D1 is less than threshold distance D2, the longer prongs will detect a higher stress from bone 88 than the shorter prong.

Reference is again made to FIG. 5. Cooling skin 22 while handheld unit 20 is in use may provide a soothing sensation to skin 22 and/or increase comfort for the user. For some applications, the cooling may be provided by a cooling element 98 that is inlaid into a transducer-side 100 of rigid transducer-skin interface 32. The inlaid nature of cooling element 98 allows a skin-side 102 of transducer-skin interface 32 to be uninterrupted by cooling element 98, e.g., to have an uninterrupted layer of the ultrasound conducting plastic with no seams between the plastic and cooling element 98, thereby preventing gel and/or dirt from getting caught in the seams and building up on skin-side 102 of transducer-skin interface 32.

For some applications, (a) a thickness T1 of the inlaid region 104 of transducer-skin interface 32, i.e., the region that is between cooling element 98 and skin-side 102 of transducer-skin interface 32, is 0.1-0.6 mm, and (b) a thickness T2 of the non-inlaid region 106 of transducer-skin interface 32, i.e., the region that is not between cooling element 98 and skin-side 102 of transducer-skin interface 32, is 0.3-2 mm. Typically, a ratio of (a) thickness T2 of non-inlaid region 106 of transducer-skin interface 32, to (b) thickness T1 of inlaid region 104 of transducer-skin interface 32 is 1-4.

For some applications, inlaid cooling element 98 may be cooled by a thermoelectric cooling element that is disposed in handheld unit 20 such that a cold side of the thermoelectric cooling element is in thermal contact with inlaid cooling element 98 (configuration not shown). Alternatively, inlaid cooling element 98 may itself be a thermoelectric cooling element.

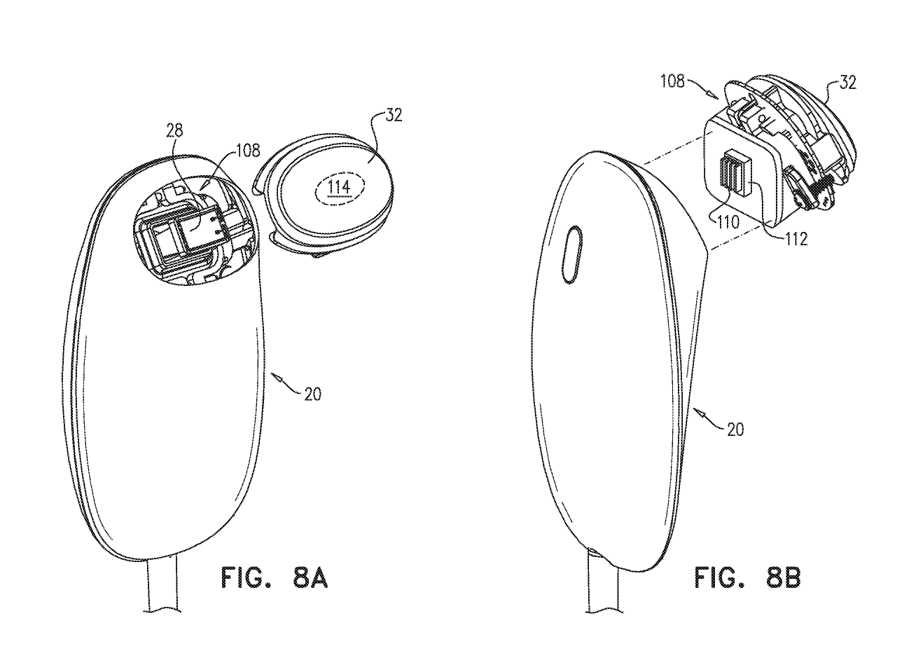

Reference is now made to FIGS. 8A-B, which are schematic illustrations of handheld unit 20 showing a chamber that houses ultrasound transducer 28, in accordance with some applications of the present invention. For some applications, handheld unit 20 is shaped to define a chamber 108 such that rigid transducer-skin interface 32 forms a side of chamber 108. Within chamber 108 is ultrasound transducer 28 and a fluid, e.g., a cooling fluid. A heat sink 110 is disposed in handheld unit 20 or on a surface of handheld unit 20 and acts to transfer heat generated by ultrasound transducer 28 to elsewhere in or around handheld unit 20. The fluid inside chamber 108 may be actively cooled by a thermoelectric cooling element 112 that is disposed in handheld unit 20 between chamber 108 and heat sink 110, such that a cold side of thermoelectric cooling element 112 is in thermal contact with chamber 108, and a hot side of thermoelectric cooling element 112 is in thermal contact with heat sink 110. Thus, thermoelectric cooling element cools the fluid and the heat generated by ultrasound transducer 28 is transferred to heat sink 110. Cooling the fluid in chamber 108 in turn cools both ultrasound transducer 28 and skin 22 through rigid transducer-skin interface 32.

For some applications, the cooling fluid inside chamber 108 may be distilled water mixed with additive materials, e.g., 40-60% ethylene glycol, or 40-60% glycerol and an antifoaming agent such as simeticone. For some applications, an ultrasound conducting gel may be used as the fluid inside chamber 108.

For some applications, a region 114 of rigid transducer-skin interface 32 that interfaces with both the fluid and skin 22 is thinner than the rest of the rigid transducer-skin interface 32 in order to increase the cooling effect on skin 22. For example, a thickness of region 114 may be 0.1-0.6 mm.

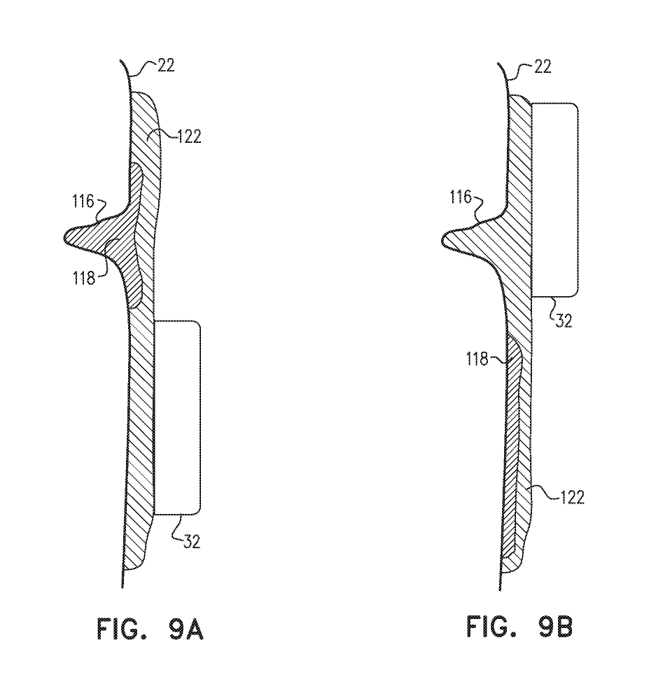

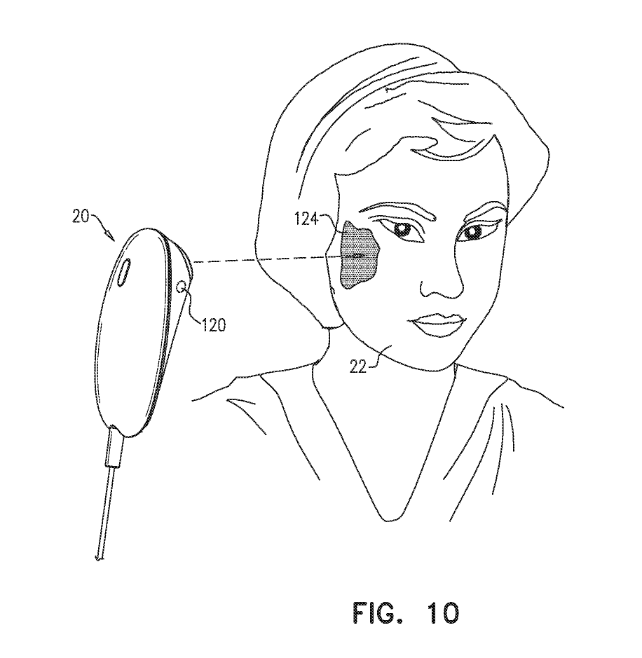

Reference is now made to FIGS. 9A-B, which are schematic illustrations of a wrinkle 116 in skin 22 of the subject, in accordance with some applications of the present invention. For some applications a viscous substance 118, e.g., a cream or gel having a viscosity in centipoise that is at least 50 times greater than the viscosity of water (e.g., 100-100,000 times greater), may be used to indicate to handheld unit 20 where ultrasound energy 30 should or should not be transmitted, and/or at what power level ultrasound energy 30 should be transmitted. A sensor 120 (FIG. 10) may be disposed in handheld unit 20 and configured to detect the presence of viscous substance 118 on skin 22 when handheld unit 20 is positioned against skin 22. Control circuitry 36 is configured to drive ultrasound transducer 28 to vary the power level of ultrasound energy 30 according to the detected presence of viscous substance 118.

For some applications, viscous substance 118 may have magnetic particles, e.g., iron particles, and sensor 120 may be a magnetic sensor that is configured to detect the presence of the magnetic particles. Alternatively, viscous substance 118 may have a dye, e.g., a visible dye or a fluorescent dye, and sensor 120 may be an optical sensor that is configured to detect the dye.

An ultrasound conducting gel 122 may be used to assist with acoustic coupling between ultrasound transducer 28 and skin 22. The user first applies viscous substance 118 to a first area on skin 22 and subsequently applies ultrasound conducting gel 122 over a second area of skin 22. For some applications, there is overlap in the areas of skin 22, such that the first area is at least partially within the second area. The viscosity of viscous substance 118 is higher, e.g., at least 1.5 times higher, than the viscosity of ultrasound conducting gel 122 such that (a) gel 122 can be applied on top of viscous substance 118 without moving viscous substance 118 around on the skin, and (b) as transducer-skin interface 32 smears gel 122 around by sliding against the skin, viscous substance 118 stays in place.

For some applications, viscous substance 118 works as a marking cream, indicating to handheld unit 20 where to transmit ultrasound energy 30. The user applies viscous substance 118 to a first area on her skin where she wants ultrasound energy 30 to be transmitted, e.g., over a wrinkle in the skin, such as is shown in FIG. 9A, and then subsequently applies ultrasound conducting gel 122 to a second area on her skin. The user then places handheld unit 20 against her skin such that ultrasound conducting gel 122 is between the skin and the handheld unit, activates control circuitry 36, and slides handheld unit 20 against her skin. For some applications, control circuitry 36 drives ultrasound transducer 28 to transmit ultrasound energy 30 through ultrasound conducting gel 122 only in response to the presence of viscous substance 118 being detected on skin 22.

Alternatively, viscous substance 118 may work as a masking cream to prevent ultrasound energy 30 from being transmitted to a specific area on skin 22. The user applies viscous substance 118 to a first area on her skin where she does not want ultrasound energy 30 being transmitted, such as is shown in FIG. 9B, and then subsequently applies ultrasound conducting gel 122 to a second area of skin. The user then places handheld unit 20 against her skin such that ultrasound conducting gel 122 is between the skin and the handheld unit, activates control circuitry 36, and slides handheld unit against her skin. Control circuitry 36 drives ultrasound transducer 28 to transmit ultrasound energy 30 through ultrasound conducting gel 122 and to terminate transmission of ultrasound energy 30 in response to the presence of viscous substance 118 being detected on skin 22.

For some applications, the detected presence of viscous substance 118 on skin 22 may indicate to control circuitry 36 to vary the power level of transmitted ultrasound energy 30. For example, control circuitry 36 may be configured to drive ultrasound transducer 28 to transmit ultrasound energy 30 at the high-power level of 20-35 W in response to the detected presence of viscous substance 118 on skin 22. Alternatively, control circuitry 36 may be configured to drive ultrasound transducer 28 to transmit ultrasound energy 30 at the low-power level of 10-20 W in response to the detected presence of viscous substance 118 on skin 22.

For some applications, viscous substance 118 may also include a skin treatment product, such as vitamins or botulinum toxin. Some other skin treatment products that may be used in viscous substance 118 are local pain killers, local anti-inflammatories, e.g., steroids or non-steroids, topical antibiotics, and/or moisturizers. It is noted that the relative thicknesses of the layers of viscous substance 118 and ultrasound conducting gel 122 as shown in FIGS. 9A-B may be exaggerated for illustrative purposes, and are not drawn to scale.

Reference is now made to FIG. 10, which is a schematic illustration of an ultrasound conducting treatment cream on skin 22 of the subject, in accordance with some applications of the present invention. For some applications, an ultrasound conducting treatment cream 124 is provided for use with skin 22. Treatment cream 124 may be configured to treat a variety of different skin conditions, e.g., peeling, and/or to achieve a variety of different effects, e.g., rejuvenation and/or moisturization. Ultrasound conducting treatment cream 124 may contain vitamins, e.g., at least one vitamin, and/or botulinum toxin. The inventors hypothesize that ultrasound energy 30 will enhance diffusion of ultrasound conducting treatment cream 124 into skin 22 when transmitted through treatment cream 124 into skin 22. For some applications, ultrasound energy 30 is transmitted through ultrasound conducting cream 124 into an epidermis layer of skin 22 to enhance diffusion of ultrasound conducting cream 124 into the epidermis.

The user may apply ultrasound conducting treatment cream 124 to an area on the skin, e.g., a wrinkle on the user's face, and then place handheld unit 20 against skin 22 such that ultrasound conducting treatment cream 124 is between skin 22 and handheld unit, i.e., between skin 22 and transducer-skin interface 32. Control circuitry 36 is then activated to drive ultrasound transducer 28 to transmit ultrasound energy 30 into skin 22 in two phases: (a) a low-power diffusion-enhancement phase, in which ultrasound transducer 28 enhances diffusion of ultrasound conducting treatment cream 124 into skin 22 by transmitting ultrasound energy 30 through ultrasound conducting treatment cream 124 into skin 22 at a first power level, and (b) a high-power therapeutic phase, in which ultrasound transducer 28 is configured to transmit ultrasound energy 30 into skin 22 at a second power level that is higher than the first power level. For example, ultrasound transducer 28 may transmit ultrasound energy 30 at a power level of 10-20 W during the diffusion-enhancement phase, and at a power level of 20-35 W during the therapeutic phase.

For some applications, the duration of the therapeutic phase is limited to less than 10 minutes per treatment area on skin 22 so as not to cause discomfort in skin 22, e.g., by avoiding excessively heating skin 22, while ultrasound transducer 28 is transmitting high-power ultrasound energy 30. However, the duration of the diffusion-enhancement phase is not limited to less than 10 minutes per treatment area, i.e., the duration of the diffusion-enhancement phase may have no limitation except for the battery life of the device, or if it has a limitation it is longer than 10 minutes per treatment are.

For some applications, handheld unit 20 has a heating element configured to apply heat at a temperature of 30-40 degrees Celsius to skin 22 in order to further enhance the diffusion of ultrasound conducting treatment cream 124 into skin 22 during the diffusion-enhancement phase. A cooling element such as described hereinabove may be used to subsequently cool the skin during the therapeutic phase.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-84. (canceled)

85. Apparatus for use with skin of a subject, the apparatus comprising:
a handheld unit;
an ultrasound transducer disposed in the handheld unit, and configured to transmit ultrasound energy into the skin,
an outwardly-facing surface of the handheld unit comprising a rigid transducer-skin interface that is configured to (a) contact the skin when the handheld unit is positioned against the skin such that the ultrasound transducer transmits the ultrasound energy through the rigid transducer-skin interface, and (b) slide against the skin,
wherein the transducer-skin interface comprises a rigid ultrasound-conducting plastic that is substantially (a) impermeable to water and (b) transparent to ultrasound energy at a transmission level of 10 J/s/cm2; and
control circuitry configured to drive the ultrasound transducer to apply the ultrasound energy to the skin.

86. The apparatus according to claim 85, wherein the rigid ultrasound conducting plastic comprises a copolyester resin.

87. The apparatus according to claim 85, wherein the rigid ultrasound conducting plastic comprises a polyamide copolymer.

88. The apparatus according to claim 85, wherein the ultrasound transducer is configured to transmit ultrasound energy into the skin at a frequency of 4-6 MHz.

89. (canceled)

90. The apparatus according to claim 85, wherein the apparatus comprises a gel in contact with the ultrasound transducer and the rigid ultrasound-conducting plastic.

91. The apparatus according to claim 85, wherein the handheld unit is shaped to define a chamber, such that the rigid transducer-skin interface comprises a side of the chamber and the ultrasound transducer is disposed within the chamber, the apparatus further comprising:
a fluid disposed within the chamber;
a heat sink disposed (a) in the handheld unit, or (b) on a surface of the handheld unit; and
a thermoelectric cooling element disposed in the handheld unit between the chamber and the heat sink, such that a cold side of the thermoelectric cooling element is in thermal contact with the chamber, and a hot side of the thermoelectric cooling element is in thermal contact with the heat sink,
the thermoelectric cooling element configured to cool the fluid, which in turn cools (a) the skin through the rigid transducer-skin interface, and (b) the ultrasound transducer.

92. (canceled)

93. The apparatus according to claim 91, wherein the fluid comprises an ultrasound conducting gel.

94. The apparatus according to claim 91, wherein a thickness of the rigid transducer-skin interface at an interface between the rigid transducer-skin interface and the fluid is 0.1-0.6 mm.

95. The apparatus according to claim 85, wherein the ultrasound transducer comprises a piezoelectric crystal assembly shaped to focus the ultrasound energy in a focal line, and the control circuitry is configured to drive the ultrasound transducer to apply the ultrasound energy to the skin such that the skin is heated in discrete lines.

96. The apparatus according to claim 95, wherein the ultrasound transducer is configured to focus the ultrasound energy at a depth of 2-3.5 mm below a surface of the skin when the transducer-skin interface is in contact with the skin.

97-99. (canceled)

100. The apparatus according to claim 95, wherein the handheld unit comprises at least one motion sensor configured to generate a signal (a) in response to the handheld unit being in motion, and (b) indicative of rotation of the handheld unit with respect to the skin, and wherein the control circuitry is configured to receive the signal in response to the handheld unit being in motion and in response thereto stop the ultrasound transducer from applying the ultrasound energy to the skin when the handheld unit is not in motion.

101. The apparatus according to claim 100, wherein the control circuitry is configured to receive the signal indicative of the rotation and in response thereto stop the ultrasound transducer applying the ultrasound energy to the skin.

102. The apparatus according to claim 100, wherein the control circuitry (a) comprises a rotation indicator and (b) is configured to receive the signal indicative of the rotation and in response thereto activate the rotation indicator to indicate to a user of the apparatus that the transducer-skin interface is rotating with respect to the skin.

103-107. (canceled)

108. The apparatus according to claim 95, wherein the control circuitry is configured to pulse the ultrasound energy such that as the transducer-skin interface slides against the skin, the ultrasound energy heats tissue under the skin in discrete lines, wherein a space between the discrete lines is 1-2 mm.

109-114. (canceled)

115. The apparatus according to claim 95, further comprising an actuator configured to move the ultrasound transducer with respect to the transducer-skin interface, and wherein the control circuitry is configured to (a) drive the ultrasound transducer to apply a first application of ultrasound energy to the skin, (b) activate the actuator to move the ultrasound transducer with respect to the transducer-skin interface, and (c) subsequently drive the ultrasound transducer to apply a second application of ultrasound energy to the skin such that the skin is heated in discrete lines with 1-2 mm of space between the discrete lines.

116. (canceled)

117. The apparatus according to claim 95, wherein the piezoelectric crystal assembly is (a) shaped as a segment of a hollow cylinder having a length of 6-10 mm along a longitudinal axis of the cylinder, and (b) shaped such that a width of the focal line is 60-100 microns.

118. The apparatus according to claim 95, wherein the piezoelectric crystal assembly comprises exactly one piezoelectric crystal shaped as a segment of a hollow cylinder having a length of 6-10 mm such that the ultrasound energy has a continuous focal line having a length of 6-10 mm.

119. The apparatus according to claim 95, wherein the piezoelectric crystal assembly is shaped as a segment of a hollow cylinder having a length of 6-10 mm, and comprises a plurality of piezoelectric crystals disposed along the length of 6-10 mm, such that the ultrasound energy has a dashed focal line having a length of 6-10 mm and comprising discrete portions.

120. (canceled)

121. A method for treating skin of a subject, the method comprising:
placing a handheld unit against the skin of the subject, the handheld unit comprising an ultrasound transducer configured to emit ultrasound energy having a focal line; and
sliding the handheld unit against the skin, such that:
while the ultrasound transducer is at a first location with respect to the skin, the ultrasound transducer heats a first discrete line of the skin by applying ultrasound energy into the skin, and
subsequently, while the ultrasound transducer is at a second location with respect to the skin 1-2 mm from the first location, the ultrasound transducer heats a second discrete line of the skin by applying ultrasound energy into the skin.

122. The method according to claim 121, further comprising moving the ultrasound transducer from the first location to the second location.

123-148. (canceled)
